(19) United States
(12) Patent Application Publication
(10) Pub. No.: US 2020/0237786 A1
(43) Pub. Date: Jul. 30, 2020

(54) DIAGNOSIS AND TREATMENT OF ENDOMETRIOSIS BY SCREENING FOR L-FORM BACTERIA

(71) Applicant: Soft Cell Biological Research, LLC, St. George, UT (US)

(72) Inventor: John Brent Hunt, St. George, UT (US)

(73) Assignee: Soft Cell Biological Research, LLC, St. George, UT (US)

(21) Appl. No.: 16/635,608

(22) PCT Filed: Aug. 1, 2018
(86) PCT No.: PCT/US2018/044885

§ 371 Date: Jan. 31, 2020

Related U.S. Application Data
(60) Provisional application No. 62/539,688, filed on Aug. 1, 2017.

(51) Int. Cl. A61K 31/65; C12Q 1/04; A61K 31/407; A61K 31/426; A61K 9/00; A61K 31/424; A61P 31/04

(52) U.S. Cl. CPC A61K 31/65; C12Q 1/04; A61K 31/407; A61P 31/04; A61K 9/0019; A61K 9/0053; A61K 31/424; A61K 31/426

(57) ABSTRACT

In some aspects, the disclosure relates to methods for screening clinical samples for the presence of L-form bacteria associated with endometriosis and/or ovarian fibroid tumors. The disclosure is based, in part, on screening methods used to diagnose a subject as likely having or likely not having endometriosis and/or an ovarian fibroid tumor. In some embodiments, the disclosure relates to methods of treating endometriosis by administering an antibiotic to a subject that has been determined to be infected by one or more strains of L-form bacteria.

### RELATED APPLICATIONS

This application is a national stage filing under 35 U.S.C. § 371 of international PCT application, PCT/US2018/044885, filed Aug. 1, 2018, which claims priority under 35 U.S.C. § 119(e) to U.S. provisional patent application, U.S. Ser. No. 62/539,688, filed Aug. 1, 2017, the entire contents of each of which are incorporated herein by reference.

### BACKGROUND

Endometriosis is a condition in which endometrium tissue abnormally grows at locations outside the uterus. Because such tissue growth often takes place on the ovaries, fallopian tubes, and surrounding tissues, the condition can cause infertility and chronic pelvic pain. Endometrial tissue will bleed as part of the menstrual cycle, and areas of endometriosis can become inflamed and scarred as a result. It is estimated that endometriosis affects 6 to 10% of women. Among women with endometriosis, about 40% will have infertility issues.

Diagnosis of endometriosis can be challenging. Although a physician may be able to feel endometrial growth during a pelvic exam, proper diagnosis cannot be confirmed by exam only. Pelvic ultrasound may identify large endometriotic cysts but will not be able to visualize and detect smaller regions of endometriosis. Laparoscopy and biopsy are presently the only methods for fully diagnosing endometriosis. These methods, however, are relatively invasive and expensive.

### BRIEF SUMMARY

In some aspects, the disclosure relates to methods for culturing and identifying L-form bacteria within a subject's sample (e.g., blood, saliva, etc.) in order to diagnose the subject as having endometriosis and/or an ovarian fibroid tumor. Such methods beneficially enable the detection of such conditions that would otherwise potentially go undetected or would require lengthier, costlier, and/or more invasive diagnosis. The methods described herein may therefore provide, in some embodiments, time savings, cost savings, as well as reductions in patient/subject anxiety associated with diagnostic uncertainty surrounding the condition.

Endometriosis may often go undetected until prolonged periods of infertility indicate that it may be an issue. Even then, an official diagnosis may require invasive laparoscopy and/or biopsy procedures. The disclosure is based, in part, on methods for detecting the presence of one or more types (e.g., species) of L-form bacterial infection within a subject that has or is suspected of having endometriosis and/or ovarian fibroid tumors. In some embodiments, the methods comprise detecting in biological samples (blood samples, etc.) taken from patients known to have endometriosis a particular subset of L-form bacteria. In some embodiments, the L-form bacteria are incapable of being cultured, and thereby detected, using standard clinical culturing protocols (e.g., standard culturing procedures for cell wall sufficient bacteria).

Determining the presence in a patient of an L-form bacteria associated with endometriosis and/or ovarian fibroid tumors can eliminate the need for such patients to undergo other more invasive and more costly diagnostic procedures. Methods described herein enable the culturing and detection of such L-form bacteria within a sample from a subject having or suspected of potentially having endometriosis (e.g., patients experiencing infertility, patients exhibiting one or more signs or symptoms or risk factors for endometriosis, etc.), providing a means for more rapidly and less invasively determining the likelihood of endometriosis before proceeding with more intensive and more costly diagnostic procedures. In addition, faster diagnoses of endometriosis can ease anxiety in patients suffering from the effects of endometriosis, particularly where the condition has proven difficult to diagnose using standard clinical measures known in the art.

Under culture conditions and process steps described herein, L-form bacteria that are associated with endometriosis and symptoms thereof can be successfully cultured and isolated, even in circumstances in which the sample from which the L-form bacteria are cultured is unable to produce any detectable growth of cell wall sufficient bacteria using conventional bacterial culturing techniques. In addition, embodiments of the disclosure have been used to culture bacteria for which no previous reports of successful culture or isolation have been made. In some embodiments, L-form bacteria having no known culturable, cell-wall-sufficient/cell-wall-including form in nature have been transformed into culturable, cell-wall-sufficient form using methods described by the disclosure.

Certain embodiments relate to methods for culturing L-form bacteria, methods of isolating a bacterial strain from a sample containing L-form bacteria, methods of transforming an unculturable L-form bacteria into a culturable cell-wall-sufficient form, and methods of analyzing a bacterial strain cultured or isolated from a sample in order to identify the bacterial strain, and/or propagate or harvest the bacterial strain.

In some aspects, the disclosure relates to methods for treating a subject having or suspected of having endometriosis. The disclosure is based, in part, on administration of one or more antibiotic agents to a subject that has been determined to be infected by one or more L-form bacteria. Accordingly, in some embodiments, the disclosure provides a method for treating endometriosis in a subject comprising obtaining (or having obtained) a biological sample from the subject; detecting (or having detected) in the biological sample one or more bacteria, each bacteria having a genus selected from Microbacterium, Bacillus, Micrococcus, and Staphylococcus; and administering to the subject an antibiotic agent based upon the presence of the one or more bacteria in the sample.

In some embodiments, a subject has or is suspected of having endometriosis. In some embodiments, a subject is characterized by one or more signs, symptoms, or risk factors associated with endometriosis. In some embodiments, a subject is a human (e.g., a female human).

In some embodiments, a biological sample is blood.

In some embodiments, detecting comprises culturing at least one L-form bacteria from a biological sample using a method described by the disclosure. In some embodiments, an L-form bacteria detected by a method described by the disclosure is not able to be cultured by conventional bacterial culture techniques. In some embodiments, an L-form bacteria detected by a method described by the disclosure is absent from a subject that does not have endometriosis or an ovarian fibroid tumor (e.g., a healthy subject).

In some embodiments, at least one of the L-form bacteria is of a species set forth in Table 2. In some embodiments, a Micrococcus bacteria is Micrococcus luteus. In some embodiments, a Bacillus bacteria is Bacillus safensis, Bacillus simplex, Bacillus licheniformis. Bacillus stratosphericus, Bacillus dretensis, Bacillus subtillis, Bacillus velezensis. Bacillus cereus, Bacillus subterraneus, Bacillus persicus, or Bacillus thuringiensis. In some embodiments, a Microbacterium bacteria is Microbacterium maritypicum.

In some embodiments, a Staphylococcus bacterium is Staphylococcus epidermis, Staphylococcus petrasii, Staphylococcus equorum, Staphylococcus pasteuri, Staphylococcus speibonae, and Staphylococcus warneri.

In some embodiments, detecting comprises detecting or having detected one or more additional L-form bacteria (e.g., one or more additional genus of L-form bacteria) in the biological sample. Examples of one or more additional L-form bacterial genera include Brachybacterium, Paenibacillus, Planococcus, Pseudomonas, Kocuria, Streptomyces, Dietzia, and Amnibacterium.

In some aspects, the disclosure relates to non-culture-based methods for detecting L-form bacteria (e.g., L-form bacteria associated with endometriosis) in a biological sample obtained from a subject. For example, in some embodiments, one or more L-form bacteria are detected in a sample using species-specific markers (e.g., identification of one or more genes, proteins, metabolites, signaling molecules, etc. that are unique to a given type, genus, and/or species of bacteria) or a panel of species-specific markers (e.g., 2, 3, 4, 5, 6, 7, 8, 9, 10, 20, or more markers). Markers or other biological, chemical or genetic characteristics could be assayed for example using hybridization assays (e.g., nucleic acid sequencing, such as DNA and RNA sequencing, sequence-specific binding assays, sequence-specific amplification, etc.) or protein binding assays, for example using antibodies (e.g., polyclonal antibodies, labeled polyclonal antibodies, monoclonal antibodies, labeled antibodies, labeled monoclonal antibodies, etc.). In some embodiments, the presence and/or identity of L-form bacteria in a biological sample are determined using nucleic acid sequencing (e.g., sanger sequencing, Illumina sequencing, nanopore sequencing, single molecule sequencing, etc.). In some embodiments, the presence and/or identity of L-form bacteria in a biological sample are determined using protein binding assays, for example an immunoassay (ELISA, radioimmunoassay, enzyme immunoassay, counting immunoassay, chemiluminescence immunoassay, fluoroimmunoassay, etc.). In some embodiments, the presence and/or identity of L-form bacteria in a biological sample are determined using an analytical method, for example mass spectrometry (MS/MS, MALDI-TOF, HPLC-MS, LC-MS, ICP-MS, Ion Mobility Spectroscopy, SELDI-TOF, etc.), a colorimetric assay, a fluorescence assay, etc.

In some embodiments, an antibiotic agent is a broad-spectrum antibiotic. In some embodiments, a broad-spectrum antibiotic is a beta-lactam antibiotic.

In some embodiments, a broad-spectrum antibiotic is a carbapenem. In some embodiments, a carbapenem is Imipenem or Meropenem.

In some embodiments, a broad-spectrum antibiotic is a tetracycline. In some embodiments, a tetracycline is Doxycycline.

In some embodiments, a broad-spectrum antibiotic is Amoxiclav.

In some embodiments, an antibiotic agent is administered to a subject intravenously. In some embodiments, an antibiotic agent is administered to a subject orally. In some embodiments, an antibiotic agent is administered to the subject during menstruation of the subject.

Additional features and advantages will be set forth in part in the description that follows, and in part will be obvious from the description, or may be learned by practice of the embodiments disclosed herein. The objects and advantages of the embodiments disclosed herein will be realized and attained by means of the elements and combinations particularly pointed out in the appended claims. It is to be understood that both the foregoing brief summary and the following detailed description are exemplary and explanatory only and are not restrictive of the embodiments disclosed herein or as claimed.

### BRIEF DESCRIPTION OF THE DRAWINGS

In order to describe various features and concepts of the present disclosure, a more particular description of certain subject matter will be rendered by reference to specific embodiments which are illustrated in the appended drawings. Understanding that these figures depict just some example embodiments and are not to be considered to be limiting in scope, various embodiments will be described and explained with additional specificity and detail through the use of the accompanying drawings in which:

FIG. 1 illustrates one embodiment of a method for screening a sample for the presence of L-form bacteria;

FIG. 2 illustrates progression of an aging cell infected with L-form bacteria;

FIG. 3 illustrates one embodiment of a method for screening for L-form bacteria including comminution of the sample;

FIG. 4 illustrates one embodiment of a method for transferring an L-form inoculant from a liquid growth medium to a solid growth medium;

FIG. 5 is a photograph of a mixed culture of Microbacterium and Staphylococcus bacteria which have been converted from an L-form morphology to a cell-wall sufficient morphology, the mixed culture showing production of a purple/violet pigment indicative of production of a biologically active agent; and

FIG. 6 is a photograph showing the individual bacterial strains of the culture of FIG. 5 after isolation into separate cultures, showing that production of the pigment has stopped.

### DETAILED DESCRIPTION

### Introduction

L-form bacteria have been found to reside in the blood and other tissues of some individuals, including individuals otherwise appearing healthy and not otherwise showing symptoms of infection. In some circumstances, such L-form bacteria may transition from a latent/asymptomatic form to a symptomatic form. The disclosure is based, in part, on detecting certain L-form bacterial infections in subjects having or suspected of having endometriosis. In some embodiments, L-form bacterial infections may go undiagnosed because standard laboratory testing procedures fail to identify L-form bacteria and fail to diagnose the subject as having an infection. Further, diagnosis of endometriosis is often not even sought until prolonged periods of infertility suggest it may be an issue.

Aspects of the disclosure relate to methods for diagnosing and/or treating a subject having or suspected of having (e.g., having a higher risk of developing relative to the normal female population) endometriosis. In some embodiments, a sample is obtained from a subject and evaluated for L-form bacteria. In some embodiments, the presence, absence and/or identity of one or more L-form bacteria in the sample is determined, for example by a culture-based method or a non-culture based method as described herein. In some embodiments, the one or more L-form bacteria of the sample are tested for antibiotic sensitivity. In some embodiments, the subject from which the biological sample was obtained is diagnosed as having or likely having (e.g., having an increased risk of developing) endometriosis and/or ovarian fibroid tumors. In some embodiments, the subject is administered a therapeutic (e.g., one or more antibiotic agents) based on the presence of the L-form bacteria and/or based on the results of antibiotic sensitivity testing. In some embodiments, a subject is treated with an antibiotic agent (e.g., is administered an antibiotic agent) that is cytostatic or cytotoxic to L-form bacteria that are present in the subject (e.g., based on detection of the L-form bacteria in the subject using one or more assays or methods described herein).

Methods are described herein which enable the isolation and culturing of L-form bacteria from a subject's sample (e.g., a biological sample, such as a blood sample). In some embodiments, methods described by the disclosure provide for the diagnosis of a subject as having an L-form bacterial infection. In some embodiments, where an L-form bacterial infection is determined to be present, the methods described herein provide for the isolation, culturing, and identification of the L-form bacteria underlying the L-form bacterial infection. In some embodiments, L-form bacteria associated with endometriosis are identified, and it can be determined that the subject has or is at risk of developing endometriosis. Such a determination may beneficially enable more effective and less costly medical care. For example, differential treatment may be provided to the subject based on the presence or absence of L-form bacteria associated with endometriosis. A positive result may allow the subject to forego other endometriosis diagnostic procedures, which are typically much more invasive and/or costly.

### Definitions

Many of the examples and embodiments described herein are described in the context of detecting the presence of, or likelihood of developing, endometriosis. However, it will be understood that the same principles may also be applied to determine the likelihood of fibroid tumors of the ovaries, and in particular, submucosal fibroid tumors of the ovaries. Thus, where the description refers to “endometriosis-associated L-form bacteria,” it will be understood that the term refers to L-form bacteria that are associated with endometriosis and/or ovarian fibroid tumors.

As used throughout this disclosure, the terms “cell-wall-sufficient bacteria” (“CWS bacteria”) or “classic-form bacteria” refer to strains of bacteria having a morphology with an identifiable and recognizable cell wall structure, such as the thick peptidoglycan layer of Gram positive bacteria and the thin peptidoglycan layer positioned between the cell membrane and the outer membrane (lipopolysaccharide layer) of Gram negative bacteria. As used herein, the term CWS bacteria also refers to mycobacteria, bacteria within the archaea domain, and other forms of bacteria known to those of skill in the art to typically exhibit a cell wall structure, even if not necessarily easily categorized as Gram positive or Gram negative bacteria.

The terms “L-form bacteria,” “pleomorphic bacteria,” “hidden bacteria,” “intracellular bacteria,” “fastidious bacteria,” and the like generally do not have standard definitions. The terms are often used synonymously, but in some instances, for example, the term “intracellular bacteria” may refer to bacteria residing within a host cell regardless of level of cell wall formation of the bacteria.

As used herein, the term “L-form bacteria” refers to strains of bacteria often found to reside intracellularly within a host cell and which do not exhibit a full cell wall structure. Such bacteria are distinguished from typical cell-wall-sufficient bacteria for which traditional culturing and detection methods are directed. “L-form bacteria” include bacterial strains with morphologies lacking any identifiable cell wall structure or cell wall components, and include strains including an undeveloped or incomplete cell wall structure, such as strains containing some cell wall components but lacking sufficient structure to fully define the cell wall (e.g., strains with variable shape as opposed to typical cocci, rod, and/or spiral characterization). The skilled artisan will recognize that the term “L-form bacteria” does not, however, refer to bacteria of the genus Mycoplasma.

In some instances, the term “L-form bacteria” therefore includes strains of bacteria that do not yet include fully recognizable cell wall structures, but which are transitioning toward cell wall sufficient strains. The term “L-form bacteria” also refers to pleomorphic bacteria which are capable of progressing from a reduced-cell-wall or absent-cell-wall-form toward a classic form with a full cell wall. The term also includes species and/or strains of bacteria that are not known to exist in nature in a CWS form, but which have been found to reside in one or more samples in L-form.

The term “L-form capable bacteria” is used herein to describe bacteria that are found within an L-form sample and which have been cultured from the L-form morphology into a CWS morphology. Such strains often exhibit flexible morphological characteristics and are able to revert back to an L-form morphology under certain environmental conditions.

Although the exemplary embodiments described herein refer specifically to bacteria, one of skill in the art will understand that certain principles disclosed herein may be utilized for culturing, screening, and/or detecting fungi (e.g., yeast), protozoans, and other pathogenic microorganisms capable of residing intracellularly within host cells and/or capable of being hidden from immune system responses within biological fluids or tissues.

As used herein, the term “sample” refers to a biological sample obtained from a subject (e.g., a subject having or suspected of having endometriosis) such as a tissue sample (e.g., endometrial tissue, cervical tissue, vaginal tissue, uterine tissue, etc.), whole blood sample, serum sample, plasma sample, urine sample, and the like. Such samples are typically obtained from mammalian sources. As used herein, “sample” may also refer to mixtures containing the tissue/clinical sample. For example, a sample may be added to or mixed with a growth medium to promote the growth of bacteria within the sample. When such a mixture is further processed (e.g., transferred, analyzed, monitored, stored, etc.), the mixture may be referred to simply as the “sample.”

### Diagnosis and Treatment of Endometriosis/Ovarian Fibroid Tumors

In some embodiments, a method for detecting an endometriosis-associated L-form bacteria within a subject in order to diagnose the subject as having endometriosis and/or an ovarian fibroid tumor includes: (1) subjecting a sample obtained from the subject to conditions that promote the culture of L-form bacteria present within the sample; (2) detecting the presence or absence of endometriosis-associated L-form bacteria as a result of the culturing; and (3) based on detecting the presence of endometriosis-associated L-form bacteria, diagnosing the subject as having endometriosis or likely having (e.g., having a higher risk of developing relative to the normal female population) endometriosis and/or an ovarian fibroid tumor.

A subject is typically a female mammal. In some embodiments, a subject is a female human. The age of a subject can vary. For example, in some embodiments, a subject is between 5 years and 85 years old. In some embodiments, a subject is between 8 years and 80 years old. In some embodiments, a subject is between 10 years and 60 years (e.g., any age between 10 and 60 years old, inclusive). In some embodiments, a subject has not undergone menopause. In some embodiments, a subject is menstruating.

Aspects of the disclosure relate to detecting one or more L-form bacteria in a subject that has or is suspected of having endometriosis and/or ovarian fibroid tumor. As used herein, “having or suspected of having endometriosis” refers to a subject characterized by one or more signs, symptoms, and/or risk factors for endometriosis. Signs and symptoms of endometriosis include but are not limited to displaced endometrial tissue, endometriomas, dysmenorrhea (painful periods), pelvic pain, cramping, pain with intercourse, pain with bowel movements or urination, menorrhagia (excessive bleeding), menometrorrhagia (bleeding between periods), infertility, fatigue, diarrhea, constipation, bloating, and nausea. Examples of risk factors for endometriosis include but are not limited to retrograde menstruation, surgical scar implantation (e.g., as a result of hysterectomy or Caesarian section), early age of onset for menstruation, delayed onset of menopause, short menstrual cycles, increased levels or exposure to estrogen, low body mass index, alcohol consumption, having one or more relatives (e.g., sister, mother, aunt, etc.) having endometriosis, having a medical condition that prevents normal passage of menstrual flow out of the body, and uterine abnormalities. Additional risk factors associated with endometriosis are described, for example by Hemmings et al. (2004) Fertility and Sterility 81(6):1513-1521.

In some embodiments, the presence or absence of L-form bacteria in a biological sample is detected relative to a control sample. A control sample is generally a biological sample obtained from a subject that is not characterized by one or more signs, symptoms, or risk factors for endometriosis. In some embodiments, a control sample is obtained from a subject that is known not to harbor L-form bacteria (e.g., a subject that is not infected with L-form bacteria). In some embodiments, a control sample is an internal control sample. An internal control sample generally refers to a biological sample obtained from a subject having or suspected of having endometriosis, where the sample is cultured according to standard microbiological culture techniques (and not L-form bacterial culture methods described by the disclosure).

Examples of standard microbiological culture techniques include direct (e.g., visual) examination of the biological sample to identify the presence of bacteria, non-selective and selective culture in liquid broth and/or agar plates, etc., and are described for example in Washington J A. Principles of Diagnosis. In: Baron S, editor. Medical Microbiology. 4th edition. Galveston (Tex.): University of Texas Medical Branch at Galveston; 1996. Chapter 10. In some embodiments, the absence of bacteria in an internal biological sample cultured by a standard microbiological culture technique and the presence of one or more L-form bacteria in a biological sample obtained from the same subject is indicative of the subject being infected with the L-form bacteria. In some embodiments, a subject infected with the L-form bacteria is diagnosed as having endometriosis or having a higher risk of developing (relative to the normal female population) endometriosis.

The disclosure is based, in part, on the detection of certain genera or species of bacteria (e.g., L-form bacteria) in biological samples obtained from a subject having or suspected of having endometriosis. In some embodiments, a subject is determined as having endometriosis based upon detection of one or more L-form bacteria selected from Microbacterium, Bacillus, Micrococcus, and Staphylococcus. In some embodiments, one or more L-form bacteria is 1, 2, 3, 4, 5, 6, 7, 8, 9, or 10 species of L-form bacteria. In some embodiments, one or more L-form bacteria is more than 10 species of L-form bacteria. Each species of the one or more L-form bacteria can vary. For example, 1 species of Microbacterium, 4 species of Bacillus, and 4 species of Staphylococcus may be detected in a biological sample. In another example a single species of Microbacterium may be detected in a biological sample. In some embodiments, at least one of the L-form bacteria is of a species set forth in Table 2.

Examples of Micrococcus bacteria include Micrococcus aloeverae, Micrococcus antarcticus, Micrococcus cohnii, Micrococcus endophyticus, Micrococcus flavus, Micrococcus lactis, Micrococcus luteus, Micrococcus lylae, Micrococcus mucilaginosis, Micrococcus roseus, Micrococcus terreus, Micrococcus mortus, and Micrococcus yunnanensis. In some embodiments the Micrococcus is Micrococcus luteus.

Examples of Bacillus bacteria include B. acidiceler, B. acidicola, B. acidiproducens, B. acidocaldarius, B. acidoterrestris, B. aeolius, B. aerius, B. aerophilus, B. agaradhaerens, B. agri, B. aidingensis, B. akibai, B. alcalophilus, B. algicola, B. alginolyticus, B. alkalidiazotrophicus, B. alkalinitrilicus, B. alkalisediminis, B. alkalitelluris, B. altitudinis, B. alveayuensis, B. alvei, B. amyloliquefaciens, B. a. subsp. amyloliquefaciens, B. a. subsp. plantarum, B. aminovorans[2], B. amylolyticus, B. andreesenii, B. aneurinilyticus, B. anthracis, B. aquimaris, B. arenosi, B. arseniciselenatis, B. arsenicus, B. aurantiacus, B. arvi, B. aryabhattai, B. asahii, B. atrophaeus, B. axarquiensis, B. azotofixans, B. azotoformans, B. badius, B. barbaricus, B. bataviensis, B. beijingensis, B. benzoevorans, B. beringensis, B. berkeleyi, B. beveridgei, B. bogoriensis, B. boroniphilus, B. borstelensis, B. brevis Migula, B. butanolivorans, B. canaveralius, B. carboniphilus, B. cecembensis, B. cellulosilyticus, B. centrosporus, B. cereus, B. chagannorensis, B. chitinolyticus, B. chondroitinus, B. choshinensis, B. chungangensis, B. cibi, B. circulans, B. clarkii, B. clausii, B. coagulans, B. coahuilensis, B. cohnii, B. composti, B. curdlanolyticus, B. cycloheptanicus, B. cytotoxicus, B. daliensis, B. decisifrondis, B. decolorationis, B. deserti, B. dipsosauri, B. drentensis, B. edaphicus, B. ehimensis, B. eiseniae, B. enclensis, B. endophyticus, B. endoradicis, B. farraginis, B. fastidiosus, B. fengqiuensis, B. firmus, B. flexus, B. foraminis, B. fordii, B. formosus, B. fortis, B. fumarioli, B. funiculus, B. fusiformis, B. galactophilus, B. galactosidilyticus, B. galliciensis, B. gelatini, B. gibsonii, B. ginsengi, B. ginsengihumi, B. ginsengisoli, B. glucanolyticus, B. gordonae, B. gottheilii, B. graminis, B. halmapalus, B. haloalkaliphilus, B. halochares, B. halodenitrificans, B. halodurans, B. halophilus, B. halosaccharovorans, B. hemicellulosilyticus, B. hemicentroti, B. herbersteinensis, B. horikoshii, B. horneckiae, B. horti, B. huizhouensis, B. humi, B. hwajinpoensis, B. idriensis, B. indicus, B. infantis, B. infernus, B. insolitus, B. invictae, B. iranensis, B. isabeliae, B. isronensis, B. jeotgali, B. kaustophilus, B. kobensis, B. kochii, B. kokeshiiformis, B. koreensis, B. korlensis, B. kribbensis, B. krulwichiae, B. laevolacticus, B. larvae, B. laterosporus, B. lautus, B. lehensis, B. lentimorbus, B. lentus, B. licheniformis, B. ligniniphilus, B. litoralis, B. locisalis, B. luciferensis, B. luteolus, B. luteus, B. macauensis, B. macerans, B. macquariensis, B. macyae, B. malacitensis, B. mannanilyticus, B. marisflavi, B. marismortui, B. marmarensis, B. massiliensis, B. megaterium, B. mesonae, B. methanolicus, B. methylotrophicus, B. migulanus, B. mojavensis, B. mucilaginosus, B. muralis, B. murimartini, B. mycoides, B. naganoensis, B. nanhaiensis, B. nanhaiisediminis, B. nealsonii, B. neidei, B. neizhouensis, B. niabensis, B. niacini, B. novalis, B. oceanisediminis, B. odysseyi, B. okhensis, B. okuhidensis, B. oleronius, B. oryzaecorticis, B. oshimensis, B. pabuli, B. pakistanensis, B. pallidus, B. pallidus, B. panacisoli, B. panaciterrae, B. pantothenticus, B. parabrevis, B. paraflexus, B. pasteurii, B. patagoniensis, B. peoriae, B. persepolensis, B. persicus, B. pervagus, B. plakortidis, B. pocheonensis, B. polygoni, B. polymyxa, B. popilliae, B. pseudalcalophilus, B. pseudofirmus, B. pseudomycoides, B. psychrodurans, B. psychrophilus, B. psychrosaccharolyticus, B. psychrotolerans, B. pulvifaciens, B. pumilus, B. purgationiresistens, B. pycnus, B. qingdaonensis, B. qingshengii, B. reuszeri, B. rhizosphaerae, B. rigui, B. ruris, B. safensis, B. salarius, B. salexigens, B. saliphilus, B. schlegelii, B. sediminis, B. selenatarsenatis, B. selenitireducens, B. seohaeanensis, B. shacheensis, B. shackletonii, B. siamensis, B. silvestris, B. simplex, B. siralis, B. smithii, B. soli, B. solimangrovi, B. solisalsi, B. songklensis, B. sonorensis, B. sphaericus, B. sporothermodurans, B. stearothermophilus, B. stratosphericus, B. subterraneus, B. subtilis, B. s. subsp. inaquosorum, B. s. subsp. spizizenii, B. s. subsp. subtilis, B. taeanensis, B. tequilensis, B. thermantarcticus, B. thermoaerophilus, B. thermoamylovorans, B. thermocatenulatus, B. thermocloacae, B. thermocopriae, B. thermodenitrificans, B. thermoglucosidasius, B. thermolactis, B. thermoleovorans, B. thermophilus, B. thermoruber, B. thermosphaericus, B. thiaminolyticus, B. thioparans, B. thuringiensis, B. tianshenii, B. trypoxylicola, B. tusciae, B. validus, B. vallismortis, B. vedderi, B. velezensis, B. vietnamensis, B. vireti, B. vulcani, B. wakoensis, B. weihenstephanensis, B. xiamenensis, B. xiaoxiensis, and B. zhanjiangensis.

In some embodiments, the Bacillus is Bacillus safensis, Bacillus simplex, Bacillus licheniformis. Bacillus stratosphericus, Bacillus dretensis, Bacillus subtillis, Bacillus velezensis. Bacillus cereus, Bacillus subterraneus, Bacillus persicus, or Bacillus thuringiensis.

Examples of Microbacterium bacteria include Microbacterium aerolatum, Microbacterium agarici, Microbacterium amylolyticum, Microbacterium aoyamense, Microbacterium aquimaris, Microbacterium arabinogalactanolyticum, Microbacterium arborescens, Microbacterium arthrosphaerae, Microbacterium aurantiacum, Microbacterium aurum, Microbacterium awajiense, Microbacterium azadirachtae, Microbacterium barkeri, Microbacterium binotii, Microbacterium chocolatum, Microbacterium deminutum, Microbacterium dextranolyticum, Microbacterium enclense, Microbacterium endophyticum, Microbacterium esteraromaticum, Microbacterium flavescens, Microbacterium flavum, Microbacterium fluvii, Microbacterium foliorum, Microbacterium ginsengisoli, Microbacterium ginsengiterrae, Microbacterium gubbeenense, Microbacterium halimionae, Microbacterium halophilum, Microbacterium halotolerans, Microbacterium hatanonis[3], Microbacterium hominis, Microbacterium humi, Microbacterium hydrocarbonoxydans, Microbacterium hydrothermale, Microbacterium immunditiarum, Microbacterium imperiale, Microbacterium indicum, Microbacterium insulae, Microbacterium invictum, Microbacterium jejuense, Microbacterium keratanolyticum, Microbacterium ketosireducens, Microbacterium kitamiense, Microbacterium koreense, Microbacterium kribbense, Microbacterium kyungheense, Microbacterium lacticum, Microbacterium lacus, Microbacterium lindanitolerans, Microbacterium liquefaciens, Microbacterium luteolum, Microbacterium luticocti, Microbacterium mangrovi, Microbacterium marinilacus, Microbacterium maritypicum, Microbacterium marinum, Microbacterium mitrae, Microbacterium murale, Microbacterium nanhaiense, Microbacterium natoriense, Microbacterium neimengense, Microbacterium oleivorans, Microbacterium oxydans, Microbacterium paludicola, Microbacterium panaciterrae, Microbacterium paraoxydans, Microbacterium petrolearium, Microbacterium phyllosphaerae, Microbacterium populi, Microbacterium profundi, Microbacterium proteolyticum, Microbacterium pseudoresistens, Microbacterium pumilum, Microbacterium pygmaeum, Microbacterium radiodurans, Microbacterium resistens, Microbacterium rhizomatis, Microbacterium saccharophilum, Microbacterium saperdae, Microbacterium schleiferi, Microbacterium sediminicola, Microbacterium sediminis, Microbacterium shaanxiense, Microbacterium soli, Microbacterium sorbitolivorans[4], Microbacterium suwonense, Microbacterium terrae, Microbacterium terregens, Microbacterium terricola, Microbacterium testaceum, Microbacterium thalassium, Microbacterium trichothecenolyticum, Microbacterium ulmi, Microbacterium xylanilyticum, and Microbacterium yannicii. In some embodiments, the Microbacterium is Microbacterium maritypicum.

Examples of Staphylococcus include S. argenteus, S. arlettae, S. agnetis, S. aureus, S. auricularis, S. capitis, S. caprae, S. carnosus, S. caseolyticus, S. chromogenes, S. cohnii, S. condimenti, S. delphini, S. devriesei, S. edaphicus, S. epidermidis, S. equorum, S. felis, S. fleurettii, S. gallinarum, S. haemolyticus, S. hominis, S. hyicus, S. intermedius, S. kloosii, S. leei, S. lentus, S. lugdunensis, S. lutrae, S. lyticans, S. massiliensis, S. microti, S. muscae, S. nepalensis, S. pasteuri, S. petrasii, S. pettenkoferi, S. piscifermentans, S. pseudintermedius, S. pseudolugdunensis, S. pulvereri, S. rostri, S. saccharolyticus, S. saprophyticus, S. schleiferi, S. schweitzeri, S. sciuri, S. simiae, S. simulans, S. stepanovicii, S. succinus, S. vitulinus, S. warneri, and S. xylosus. In some embodiments, a Staphylococcus bacterium is Staphylococcus epidermis, Staphylococcus petrasii, Staphylococcus equorum, Staphylococcus pasteuri, Staphylococcus speibonae, and Staphylococcus warneri.

In some embodiments, detecting comprises detecting or having detected one or more additional L-form bacteria (e.g., genus of L-form bacteria that are not Microbacterium, Bacillus, Micrococcus, and Staphylococcus) in the biological sample. Examples of one or more additional L-form bacterial genera include Brachybacterium, Paenibacillus, Planococcus, Pseudomonas, Kocuria, Streptomyces, Dietzia, and Amnibacterium. In some embodiments, the one or more additional L-form bacteria is a species listed in Table 2.

In some embodiments, the presence of L-form bacteria in a biological sample is detected and/or species of L-form bacteria are identified after performing a method of L-form bacterial culture described by the disclosure. In some embodiments, L-form bacteria that have been cultured according to a method described herein are identified by phenotypic analysis (e.g., morphology), for example after visual analysis under a microscope. In some embodiments, an L-form bacteria is identified using a genetic method, for example nucleic acid sequencing such as 16S rRNA sequencing. In some embodiments, an L-form bacteria is identified using biochemical assays, for example Gram-staining or by chemotaxonomic methods such as analysis of quinone system or fatty acid profiles, or by restriction enzyme digestion analysis.

In some embodiments, the detecting comprises a non-culture-based method (e.g., a method that does not require culturing of bacterial cells from the biological sample prior to analysis) for detecting L-form bacteria (e.g., L-form bacteria associated with endometriosis). In some embodiments, one or more L-form bacteria are detected in a sample using species-specific markers (e.g., identification of one or more genes, proteins, metabolites, signaling molecules, etc. that are unique to a given type, genus, and/or species of bacteria). In some embodiments, one or more species-specific marker is detected using a hybridization assay, for example nucleic acid sequencing (e.g., DNA or RNA sequencing), such as direct 16S rRNA gene sequencing. In some embodiments, the presence and/or identity of L-form bacteria in a biological sample are determined using an analytical method, for example by matrix assisted laser desorption ionization time of flight (MALDI-TOF) mass spectrometry.

Some embodiments may further include providing differential treatment based on the results of the foregoing steps. In some circumstances, where a patient is identified as harboring one or more endometriosis-associated L-form bacteria, the likelihood of endometriosis being at least part of the patient's symptoms may be determined to be likely, and further testing for endometriosis (e.g., biopsy and/or laparoscopy) may be justified. On the other hand, where no endometriosis-associated L-form bacteria are detected, a different treatment protocol, such as first testing for other potential causes of the patient's symptoms (e.g., hormone level measurements) may be called for prior to trying a more invasive procedure.

Such differential treatment beneficially allows for a more focused treatment and testing strategy, thereby saving time and money as well as potentially lessening patient anxiety and discomfort. In at least some circumstances, the detection of endometriosis-associated L-form bacteria allows other potential causes of the patient's symptoms to be ruled out or at least determined as less likely. For example, as described above, where endometriosis-associated bacteria are detected, endometriosis may be at least provisionally determined as the most likely cause of the patient's symptoms, and further testing and/or treatment may proceed accordingly. Alternatively, where no endometriosis-associated L-form bacteria are detected within the patient, the patient may be considered as likely not having such endometriosis, and may be subjected to a different line of further testing in an attempt to identify a different cause of endometriosis or to determine some other cause of underlying symptoms.

In some circumstances, a patient may be tested prior to experiencing any infertility issues or other symptoms of endometriosis. For example, as a prophylactic measure, a patient may be tested to determine the presence or absence of endometriosis-associated L-form bacteria and thus the likelihood that the patient has or may in the future develop endometriosis.

As described herein, the presence of certain L-form bacteria is correlated to, and may be causally linked to, endometriosis and/or fibroid tumors of the ovaries. Using the methods described herein, it has been found that the presence in a patient's sample of certain L-form bacteria, for example L-form bacteria of the genus Microbacterium, highly correlates with the patient having endometriosis and/or fibroid tumors of the ovaries. In some embodiments, the species of Microbacterium is Microbacterium maritypicum. As described further in the Examples, of females tested which were found to harbor an L-form of Microbacterium maritypicum, more than 80% had previously indicated that they suffered from endometriosis or fibroid tumors of the ovaries.

In some embodiments, a patient may harbor an L-form Microbacterium strain along with at least one other antagonist or symbiont L-form bacterial strain. In some embodiments, a method for diagnosing the patient as having endometriosis and/or an ovarian fibroid tumor includes: (1) subjecting a sample obtained from the patient to conditions that promote the culture of L-form bacteria present within the sample; (2) detecting the presence of Microbacterium L-form bacteria, such as Microbacterium maritypicum, as a result of the culturing; (3) detecting the presence of at least one other L-form bacteria strain that is an antagonist or symbiont with the Microbacterium L-form bacteria; and (4) based on detecting the presence of the Microbacterium L-form bacteria and the at least one other L-form bacteria strain, diagnosing the patient as having or likely having endometriosis and/or an ovarian fibroid tumor. In some embodiments, the at least one other L-form bacteria is a Bacillus or Staphylococcus strain.

In some embodiments, the Microbacterium and the at least one other L-form bacteria interact with one another within the host patient to produce one or more biologically active agents that are correlated with or causally linked to the patient's symptoms. Thus, in some embodiments, a method for diagnosing a patient as having endometriosis and/or an ovarian fibroid tumor includes testing a sample obtained from the patient for the sufficient presence of one or more biologically active agents produced by the mixed L-form culture.

The disclosure relates, in some aspects, to methods for treating endometriosis or ovarian fibroid tumor in a subject in need thereof. In some embodiments, a subject in need thereof is a subject who 1) exhibits one or more signs, symptoms, or risk factors for endometriosis, and/or 2) has been determined to harbor an L-form bacterial infection according to culture methods described by the disclosure. In some embodiments, a method of treating endometriosis comprises administering a subject one or more antibiotic agents.

As used herein an “antibiotic agent” refers to a small molecule, protein, peptide, polypeptide, or nucleic acid that inhibits growth of or destroys (e.g., kills or results in death of) a microorganism (e.g., bacteria, fungi, protozoans, yeast, etc.). In some embodiments, an antibiotic agent (e.g., an antibacterial agent) is an agent that is effective in killing or inhibiting growth of L-form bacteria.

In some embodiments, an antibiotic agent is a broad-spectrum antibiotic (e.g., antibiotics that are effective against both Gram-positive and Gram-negative bacteria), an antibiotic that inhibits bacterial cell wall growth (e.g., carbapenems, beta-lactam antibiotics, glycopeptides, penicillins, etc.), or an antibiotic that inhibits DNA or RNA synthesis or function (e.g., aminoglycosides, macrolides, quinolones, tetracyclines, etc.). Examples of antibiotic (antibacterial) agents include aminoglycosides, ansamycins, carbacephems, carbapenems, cephalosporins, glycopeptides, lincosamides, lipopeptides, macrolides, monobactams, nitrofurans, oxazolidinones, penicillins, antibacterial polypeptides, quinolones and fluoroquinolones, sulfonamides, tetracyclines, and other agents (e.g., Clofazimine, Dapsone, Capreomycin, Cycloserine, Ethambutol, Ethionamide, Isoniazid, Pyrazinamide, Rifampicin, Rifabutin, Rifapentine, Streptomycin, Arsphenamine, Chloramphenicol, Fosfomycin, Fusidic acid, Metronidazole, Mupirocin, Platensimycin, Quinupristin/Dalfopristin, Thiamphenicol, Tigecycline, Tinidazole, Trimethoprim, etc.).

In some embodiments, an antibiotic agent is an aminoglycoside. Examples of aminoglycosides include but are not limited to Amikacin, Gentamicin, Kanamycin, Neomycin, Netilmicin, Tobramycin, Paromomycin, Streptomycin, and Spectinomycin.

In some embodiments, an antibiotic agent is an ansamycin. Examples of ansamycins include but are not limited to Geldanamycin, Herbimycin, and Rifaximin.

In some embodiments, an antibiotic agent is a carbaphem (e.g., Loracarbef) or a carbapenem. Examples of carbapenems include but are not limited to Ertapenem, Doripenem, Imipenem (or Imipenem/Cilastatin), and Meropenem.

In some embodiments, an antibiotic agent is a cephalosporin. Examples of cephalosporins include but are not limited to Cefadroxil, Cefazolin, Cephradine, Cephapirin, Cephalothin, Cefalexin, Cefaclor, Cefoxitin, Cefotetan, Cefamandole, Cefmetazole, Cefonicid, Loracarbef, Cefprozil, Cefuroxime, Cefixime, Cefdinir, Cefditoren, Cefoperazone, Cefotaxime, Cefpodoxime, Ceftazidime, Ceftibuten, Ceftizoxime, Moxalactam, Ceftriaxone, Cefepime, Ceftaroline fosamil, and Ceftobiprole.

In some embodiments, an antibiotic agent is a glycopeptide. Examples of glycopeptides include but are not limited to Teicoplanin, Vancomycin, Telavancin, Dalbavancin, and Oritavancin.

In some embodiments, an antibiotic agent is a lincosamide. Examples of lincosamides include but are not limited to Clindamycin and Lincomycin.

In some embodiments, an antibiotic agent is a lipopeptide, for example Daptomycin.

In some embodiments, an antibiotic agent is a macrolide. Examples of macrolides include but are not limited to Azithromycin, Clarithromycin, Erythromycin, Roxithromycin, Telithromycin, and Spiramycin.

In some embodiments an antibiotic agent is a monobactam, for example Azitreonam.

In some embodiments, an antibiotic agent is a nutrofuran. Examples of nitrofurans include but are not limited to Furacolidone and Nutrofurantoin.

In some embodiments, an antibiotic agent is an oxazolidinone. Examples of oxaxolidinones include but are not limited to Linezolid, Posizolid, Radezolid, and Torezolid.

In some embodiments, an antibiotic agent is a penicillin or a penicillin derivative. Examples of penicillin or penicillin derivatives include but are not limited to Amoxicillin, Ampicillin, Azlocillin, Dicloxacillin, Flucloxacillin, Mezlocillin, Methicillin, Nafcillin, Oxacillin, Penicillin G, Penicillin V, Piperacillin, Penicillin G, Temocillin, and Ticarcillin. In some embodiments, an antibiotic agent is a penicillin combination, for example Amoxicillin/clavulanate, Ampicillin/sulbactam, Piperacillin/tazobactam, and Ticarcillin/clavulanate.

In some embodiments, an antibiotic agent is an antibacterial polypeptide, for example Bacitracin, Colistin, or Polymyxin B.

In some embodiments, an antibiotic agent is a quinolone or a fluoroquinolone. Examples of quinolones and fluoroquinolones include but are not limited to Ciprofloxacin, Enoxacin, Gatifloxacin, Gemifloxacin, Levofloxacin, Lomefloxacin, Moxifloxacin, Nadifloxacin, Nalidixic acid, Norfloxacin, Ofloxacin, Trovafloxacin, Grepafloxacin, Sparfloxacin, and Temafloxacin.

In some embodiments, an antibiotic agent is a sulfonamide. Examples of sulfonamides include but are not limited to Mafenide, Sulfacetamide, Sulfadiazine, Silver sulfadiazine, Sulfadimethoxine, Sulfamethizole, Sulfamethoxazole, Sulfanilimide, Sulfasalazine, Sulfisoxazole, Trimethoprim-Sulfamethoxazole (Co-trimoxazole; TMP-SMX), and Sulfonamidochrysoidine.

In some embodiments, an antibiotic agent is a tetracycline. Examples of tetracyclines include but are not limited to Demeclocycline, Doxycycline, Metacycline, Minocycline, Oxytetracycline, and Tetracycline.

The disclosure is based, in part, on the recognition that certain L-form bacteria are sensitive to (e.g., not resistant to treatment with) specific antibiotic agents. In some embodiments, resistance to antibiotic agents is tested by a diffusion tab (diffusion disk) test, for example as described in Reller et al. (2009) Clinical Infectious Diseases, 49(11): 1749-1755. However, the skilled artisan recognizes that additional methods of antibiotic sensitivity testing may also be used. In some embodiments, an L-form bacteria associated with endometriosis (e.g., Microbacterium maritypicum) is sensitive to carbapenem antibiotic agents and/or tetracycline antibiotic agents, for example Imipenem, Meropenem, Doxycycline, or any combination thereof.

The route of administration to a subject may vary. In some embodiments, an antibiotic agent is administered to a subject intravenously. In some embodiments, an antibiotic agent is administered to a subject orally. In cases where multiple (e.g., 2, 3, 4, 5, 6, 7, 8, 9, or 10) antibiotic agents are administered to a subject, each antibiotic agent may be administered via the same route or different routes.

The dose and frequency of administration of an antibiotic agent to a subject may vary. Generally, a subject is administered a therapeutically effective amount of an antibiotic agent. A “therapeutically effective amount” generally refers to the amount of an antibiotic agent that is capable of producing a desired result (e.g., reduction of severity or frequency of one or more signs or symptoms of endometriosis, and/or reduction or clearance of an L-form bacterial infection).

In some embodiments, an antibiotic agent is administered to a subject prior to the beginning of the subject's menstrual cycle (e.g., after the end of one period of menstrual bleeding and prior to the beginning of the next period of menstrual bleeding). In some embodiments, a subject is administered an antibiotic agent 1, 2, 3, 4, 5, 6, 7, 8, 9, or 10 days prior to the beginning of the subject's menstrual cycle. In some embodiments, a subject is administered an antibiotic agent during menstruation (e.g., while menstrual bleeding is occurring). In some embodiments, a subject is administered an antibiotic agent both prior to and during the subject's menstrual cycle. In some embodiments, an antibiotic agent is not administered to a subject while menstrual bleeding is not occurring (e.g., in between menstruation of the subject). In some embodiments, an antibiotic agent is administered to a subject (e.g., administered once, twice, three times, four times, etc., per day) for between 1 week and 6 months, between 3 weeks and 10 months, or between 6 months and 12 months, for example through 1, 2, 3, 4, 5, 6, 7, 8, 9, 10, 11, 12, or more menstrual cycles. In some embodiments, an antibiotic agent is administered to a subject for between 1, 2, 3, 4, 5, 6, 7, 8, 9, 10, or more years.

In some embodiments, the duration of the treatment (e.g., administration of an antibiotic agent to the subject) is determined by the persistence of the L-form bacterial infection. In some embodiments, a subject is evaluated after treatment (e.g., monthly, every six months, or at an suitable frequency) to determine whether the L-form bacterial infection has been cleared. In some embodiments, treatment is continued until there are no longer any detectable signs of L-form bacterial infection in a sample (e.g., in a blood or other sample described in this application, for example using a cell growth assay and/or a molecular detection assay) obtained from a subject during treatment. In some embodiments, treatment is continued for a while (e.g., for a week, a month, 1-6 months, or longer) after there are no longer any detectable signs of L-form infection in sample obtained from the subject during (or after cessation of) treatment.

Compositions for different routes of administration are well known in the art (see, e.g., Remington's Pharmaceutical Sciences by E. W. Martin).

Dosage will depend on the subject and the route of administration. Dosage can be determined by the skilled artisan. In some embodiments, a subject is administered a dose of an antibiotic agent that is consistent with the dosing guidelines set forth in Porter, R. S., Kaplan, J. L., & Merck & Co. (2018). The Merck manual of diagnosis and therapy. Whitehouse Station, N.J: Merck Sharp & Dohme Corp. For example, in some embodiments, a subject is orally administered an antibiotic agent at a dosage between about 1 mg and about 2500 mg per dose (e.g., any integer between 1 and 2500, inclusive) and a frequency of between 1 time and 5 (e.g., 1, 2, 3, 4, or 5) times per day. In some embodiments, a subject is orally administered an antibiotic agent at a dosage between about 10 mg and about 250 mg per dose and a frequency of between 1 time and 5 times per day. In some embodiments, a subject is orally administered an antibiotic agent at a dosage between about 100 mg and about 1000 mg per dose and a frequency of between 1 time and 5 times per day. In some embodiments, a subject is intravenously (IV) administered an antibiotic agent at a dosage between about 500 mg and about 5 g per four to six hours. In some embodiments, a subject is intravenously (IV) administered an antibiotic agent at a dosage between about 1 g and about 5 g per six to eight hours, 500 mg once per day (24 hours), 1 and 3 grams every eight hours, etc.

The disclosure relates, in some aspects, to administration of one or more antibiotic agents to a subject to treat endometriosis. As used herein, the terms “treat” and “treating” mean to reduce the severity or frequency of at least one sign or symptom of a disease or disorder. For example, treating endometriosis, in some embodiments, relates to the reduction of one or more signs or symptoms of endometriosis, such as displaced endometrial tissue, endometriomas, dysmenorrhea (painful periods), pelvic pain, cramping, pain with intercourse, pain with bowel movements or urination, menorrhagia (excessive bleeding), menometrorrhagia (bleeding between periods), infertility, fatigue, diarrhea, constipation, bloating, and nausea. In some embodiments, treating endometriosis refers to the reduction (e.g., reduction of about 50%, 60%, 70%, 80%, 90%, 99%, etc.) or clearance (e.g., total killing or removal from a subject such that there is an undetectable level of L-form bacteria relative to the level of L-form bacteria in a subject prior to treatment) of one or more L-form bacteria that are associated with endometriosis.

### Methods of Screening for L-form Bacteria

In some embodiments, methods of culturing L-form bacteria are described by US Patent Publication US2016/0168614, the entire contents of which are incorporated herein by reference.

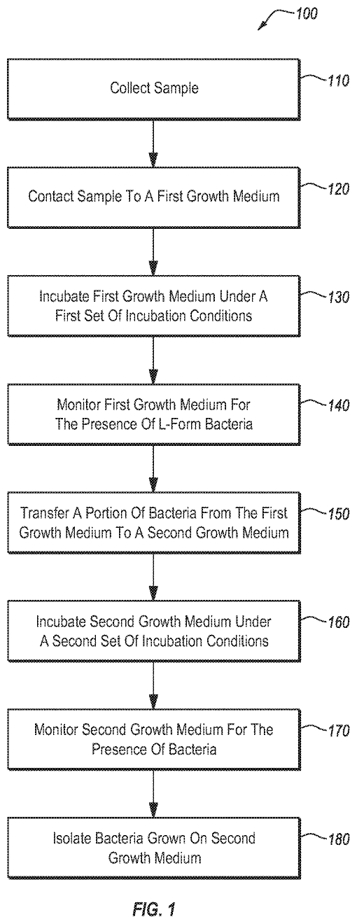

FIG. 1 illustrates an exemplary method 100 of screening a sample for the presence of L-form bacteria and culturing/producing L-form capable bacteria. In some embodiments, the method includes a step 110 of collecting a sample, a step 120 of contacting the sample to a first growth medium, a step 130 of incubating the inoculated first growth medium under a first set of incubation conditions, and a step 140 of monitoring the inoculated first growth medium for the presence of L-form bacteria.

In some embodiments, the step 110 of collecting the sample is performed using standard sample collection techniques, such as a blood draw, tissue swab, plant tissue extraction, and the like. In some embodiments, the sample is collected in the same container in which the first growth medium is contained. Alternatively, the sample may be collected in one or more separate containers prior to storage, transport, and subsequent transfer to the container holding the first growth medium.

Various types and/or combinations of growth media may be used as the first growth medium. For example, the first growth medium may be formulated as complex growth media (e.g., blood, yeast extract, bile, peptone, serum, and/or starch containing medias), defined growth media, or a selective media (e.g., nutrient selective for mannitol, cysteine, lactose, sucrose, salicin, xylose, lysine, or combinations thereof; selective based on carbon source, nitrogen source, energy source, and/or essential amino acids, lipids, vitamins, minerals, trace elements, or other nutrients; and/or selective antibiotic/antimicrobial containing media). Exemplary growth media that may be used in solid (e.g., with agarose) or liquid form include R2A, nutrient, chocolate blood, blood, mannitol salt, Vogel Johnson, Kligler iron, Simmons citrate, Columbia, cetrimide, xylose-lysine-deoxycholate, tryptic soy, Tinsdale, Phenylethyl alcohol, Mueller-Hinton, MacConkey, brain-heart infusion (BHI), and lysogeny broth media.

In preferred embodiments, the first growth medium is a liquid growth medium. In one particular example, the growth media is serum (e.g., human, bovine) and/or brain-heart infusion (BHI) broth, and may be contacted with the sample as a liquid in suspension with the sample. In preferred embodiments the growth media is formulated without substances that would hamper or restrict the growth of any bacteria found within the sample. For example, the growth media preferably omits antimicrobial enzymes (e.g., lysozyme, protease, etc.), antimicrobial peptides, and immune system components (e.g., leukocytes, complement system proteins, antibodies or other immunoglobulins, etc.).

For example, it has been discovered that L-form bacteria are often able to reside within a sample at a low-grade level without eliciting a full immune response and without converting to classic form. The presence of immune system components or other growth hampering substances within such samples can prevent the bacteria from being manifest in classic form, even though the bacteria are present within the sample in L-form. Under such circumstances, the removal or dilution of growth hampering substances and/or the transfer of L-form bacteria to growth media without growth hampering substances can promote progression of the bacteria within the sample to classic form, and thereby provide faster culture and screening of L-form bacteria within the sample.

In some embodiments, the first set of incubation conditions promote the aging of the sample tissue cells, allowing L-form bacteria present within the cells to grow. For example, as the cells die and rupture, more L-form bacteria are able to escape their intracellular positions and move into the surrounding extracellular medium. In addition, the dilution of the sample within the first growth medium dilutes the concentration of antibodies and other immune system components present within the blood sample, also enabling greater growth of the L-form bacteria.

In some embodiments, immune system components may be removed from the sample or from the inoculated first growth medium, or can be inactivated by adding an inactivating agent, such as a binding compound or complement inactivator, by adding one or more blocking antibodies, by washing, centrifuging, and/or filtering the sample to separate cells from other immune system molecules, or simply by diluting the sample sufficiently within the growth medium to render the components ineffective. In preferred embodiments, however, substances that would hamper polymerase chain reaction (PCR) or other analysis techniques (such as ethylenediaminetetraacetate (EDTA)), or that would inhibit conversion/reversion to classic form (such as EDTA), are omitted.

After the sample is contacted with the first growth medium in step 120, the method proceeds to step 130 by incubating the inoculated first growth medium under a first set of incubation conditions. The collected sample is stored at a temperature about body temperature or at a temperature lower than about body temperature. For example, the collected sample may be stored at a temperature, constant or fluctuating, within a range or about 20° C. to about 40° C., or within a range of about 25° C. to about 35° C., or more preferably within a range of about 25° C. to about 30° C., or about 27° C. In preferred embodiments, the inoculated first growth medium is stored at a temperature below body temperature.

It has been surprisingly found that L-form bacteria within a sample grow at a greater rate at temperatures lower than body temperature. For example, in human blood samples, which are typically stored at body temperature (37° C.), it has been found that storage at a lower temperature increases the growth of L-form bacteria within the sample and enables L-form bacteria, which would otherwise remain present at non-detectable levels, to grow to observable levels. Preferably, incubation also omits rocking or shaking of the growth medium in order to reduce the amount of contact between any L-form bacteria and any antibodies or other immune components that may be present within the sample.

The inoculated first growth medium is incubated for a time sufficient to provide growth of any L-form bacteria present within the sample (e.g., for a time sufficient to allow any L-form bacteria present within the sample to achieve a detectable population). In some embodiments, this monitoring period can be about 120 hours or even longer than 120 hours. In more preferred embodiments, this monitoring period can be less than about 120 hours. For example, in some embodiments, the monitoring period can be within a range of about 24 hours to about 96 hours, or within a range of about 36 hours to about 84 hours. In other embodiments, the monitoring period is within a range of about 48 hours to about 72 hours.

In some embodiments, a dual track culturing setup is followed by subjecting a first set of sample portions to a short-track monitoring period and a second set of sample portions to a long-track monitoring period, where the short and long-track monitoring periods have durations according to the above ranges, with the short-track duration being shorter than the long-track duration. Such a dual-track setup has shown good results by enabling faster results from the short-track, when possible, without missing the detection of other, slower species and/or strains resulting from the long-track.

Step 140 of monitoring the first growth medium during the monitoring period for the presence of any L-form bacteria may be performed by transferring the sample or a portion of the stored sample to a microscope slide, well plate, or other such apparatus allowing the microscopic visualization of the sample or portion of the sample. In preferred embodiments, in order to avoid the disruption of potentially fragile L-form bacteria within the sample or portion of the sample collected for microscopic inspection, the visual monitoring is carried out without traditional staining (e.g., Gram staining) or chemical or heat fixing steps. For example, the visual monitoring may be carried out by direct microscopic observation of the sample or portion thereof by preparing a wet-mount, live slide for observation. Although microscopy using live slides is the preferred manner of monitoring for L-form growth, other suitable monitoring techniques include spectrophotometric methods (including colorimetry and measurement of optical density), staining, and measurements of turbidity, total cellular DNA and/or protein levels, electrical field impedance, bioluminescence, carbon dioxide, oxygen, ATP production or consumption, and the like.

Monitoring of the first growth medium may be carried out throughout the monitoring period. For example, monitoring may occur periodically according to a set schedule throughout the monitoring period, such as at set intervals (e.g., daily, every 12 hours, every 10, 8, or 6 hours, every 4, 3, or 2 hours, hourly, or even more frequently). In some circumstances, a sample may be monitored throughout a monitoring period, and may fail to exhibit any indication of bacterial presence. At this point, in some embodiments, the method is completed and a negative result is returned (e.g., the method either detected or failed to detect the presence of any L-form bacteria in the sample).

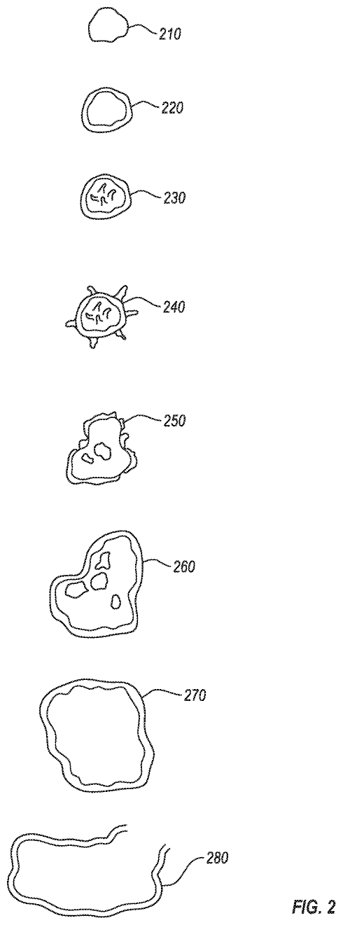

Prior to transferring to a second growth medium, the inoculated first growth medium is preferably incubated until L-form bacteria within the medium have progressed to a state of sufficient growth. FIG. 2 illustrates a typical progression of a red blood cell harboring L-form bacteria once placed under the first set of incubation conditions. A healthy red blood cell 210 that harbors L-form bacteria will begin to progress to a first state 220, where internal pressure is created by developing L-form bacteria within the cell. At a second state 230, L-form bacteria begin to transition from a non-microscopically observable form (e.g., under about 0.05 μm) to an observable form. At a third state 240, internal structures of the red blood cell begin to break down (e.g., through the action of lysozymes), freeing up additional nutrients for L-form growth and creating greater internal pressure within the cell. In some circumstances it has been observed that many cells stay at this state for long periods of time (e.g., several weeks or months). L-form bacteria appear to be present in such cells, but the L-form bacteria are not released from the cells at detectable levels. When these types of cells are present, embodiments utilizing a comminuting step may be particularly advantageous.

In other circumstances, cells continue toward more progressed states. At a fourth state 250, outward protrusions of the cell become visible through weak spots in the wall of the degrading red blood cell. At a fifth state 260 and a sixth state 270, the cell wall further breaks down and the cell continues to expand toward its limits. At a seventh state 280, the cell ruptures due to degradation and excessive internal bacterial growth, releasing L-form bacteria into the surrounding growth medium.

Preferably, the inoculated first growth medium is incubated until at least some (e.g., 10% or more, 25% or more, 50% or more, 75% or more, 90% or more) of the monitored cells of the sample have progressed to a state where they have ruptured to release intracellular L-form bacteria.

Some embodiments further include a step 150 of transferring at least a portion of the inoculated first growth medium to a second growth medium, and a step 160 of incubating the second growth medium under a second set of incubation conditions. In preferred embodiments, the second growth medium is a solid-phase growth medium (e.g., contained in a plate or slant). For example, solid-phase growth media may include one or more of the growth media described above (e.g., complex media, defined media, minimal or selective media) incorporated into a solid substrate. Suitable solid substrates include those formed with agarose, collagen, laminin, elastin, peptidoglycan, fibronectin, and the like.

The second set of incubation conditions includes a temperature within a range of about 20° C. to about 40° C. Preferably, the second growth medium is incubated at approximately body temperature (about 30° C. to 40° C. or about 37° C.). The second growth medium is incubated at this temperature for a time period of about 24 hours to 96 hours, or about 36 hours to 84 hours, or about 48 hours to 72 hours, or about 60 hours. In some embodiments, the temperature is then adjusted to a range that is below body temperature (e.g., about 25° C. to 35° C., or about 25° C. to 30° C., or about 27° C.) for a time period of about 4 days to about 30 days, or about 7 days to about 21 days, or about 14 days. In preferred embodiments, the temperature is adjusted to a range that is below body temperature for a time period of about 1-7 days, or about 3-5 days.

In some embodiments, the step 150 includes transfer to multiple types of solid-phase growth media in order to isolate multiple strains that may be present within the sample. For example, a set of agar plates may be prepared to receive the sample, with several of the agar plates containing different forms of media (such as any of those types discussed above with respect to the sample collection device, including selective growth media), and these may be further divided by placing one set under aerobic conditions after inoculation and another set under anaerobic conditions after inoculation (e.g., by placing in a standard anaerobic chamber maintained with carbon dioxide). During or after incubation, the method can include the step 170 of monitoring the second growth medium for bacterial growth (e.g., using one or more of the monitoring techniques described herein).

Although defined medias may be used as growth media in the methods described herein, it has been found that L-form bacteria are able to be efficiently cultured and detected using various complex medias such as BHI medias or those including serum (as the first and/or second growth medias). Beneficially, the methods described herein have enabled the screening of L-form bacteria without the need for generally more expensive defined medium formulations. Without being bound to any particular theory, it is thought that one or more process steps, such as the particular incubation conditions (e.g., time, temperature) and/or transfer steps (e.g., transferring bacteria in a manner that enables bacteria within a sample to maintain a hydrated state) enables L-form bacteria to be cultured without the need for custom-made or defined medias.

Referring back to FIG. 1, some embodiments further include a step 180 of isolating bacteria grown on the second growth medium. As growth occurs on the second growth medium, some strains of L-form bacteria may transition to classic form morphologies and may grow classic form colonies on the second growth medium. Such bacteria may be transferred to separate media (e.g., one or more complex, selective, or defined medias described herein) until a single strain is found on the media, and/or may be sampled and further analyzed according to well-known microbiological characterization techniques, including microscopic examination, staining (e.g., Gram, Malachite green/Safranin, and acid-fast stains), and selective growth testing. Other analytical techniques such as chromatography, gel separation, immunoassays, flow-through assays (e.g., plasmon resonance detection), fluorescent probe binding and measurement, automated cell/plate counting, microwell reading, DNA hybridization and amplification methods (e.g., polymerase chain reaction, strand displacement amplification), 16S rDNA sequencing, other molecular biological characterization techniques, and combinations thereof may also be used to analyze bacteria cultured or isolated using the methods described herein.

Beneficially, many of the bacteria cultured to a CWS form using one of the culturing embodiments described herein maintain a flexible morphology capable of reverting back to L-form when exposed to appropriate conditions.

Any of the foregoing embodiments may also include the addition of a transmembrane protein inhibiting agent to promote and/or augment the conversion of L-form bacteria to CWS form. It is presently theorized that at least some L-form bacteria are capable of resisting a host immune response by modulating or secreting one or more of the host's transmembrane proteins in order to inhibit or dampen the host's immune response. In one example, an L-form bacteria may modulate or secrete the CD47 protein in order to inhibit macrophage response as a result of the CD47 protein engaging with SIRP-α. It is presently believed that inhibiting one or more of such transmembrane proteins in a collected sample (e.g., blood sample) will trigger or augment the conversion of L-form bacteria to CWS form. Some embodiments may therefore include the removal of one or more transmembrane proteins and/or the addition of an inhibiting agent (e.g., a targeted antibody) to inhibit one or more transmembrane proteins in order to trigger or augment the conversion of L-form bacteria to CWS form.

### Sample Comminution

In some circumstances, it may be desirable to subject a sample to blending, vortexing, sonication, or other disruptive processes or combinations thereof in order to disassociate biofilms and/or aggregates, to rupture cells, or to otherwise disperse any bacteria and increase exposure to surrounding growth media (e.g., prior to culturing of L-form bacteria or further screening). It has been surprisingly found that proper use of a comminution step in a screening process can increase yields, reduce culture times, and allow for faster detection and screening of samples having L-form bacteria. In some embodiments, a biological sample (e.g., cells of a biological sample) are subjected to one or more techniques to lyse cells containing L-form bacteria (e.g., release intracellular L-form bacteria into culture media), for example by subjecting the sample (or cells) to one or more freeze-thaw cycles or other cell lysis techniques known in the art. Although the exemplary method may be used to prepare any of the forms of samples defined above, it may be particularly useful in preparing samples known to contain, or known to be likely to contain, biofilms, root nodules, and/or other aggregates potentially harboring L-form growth.

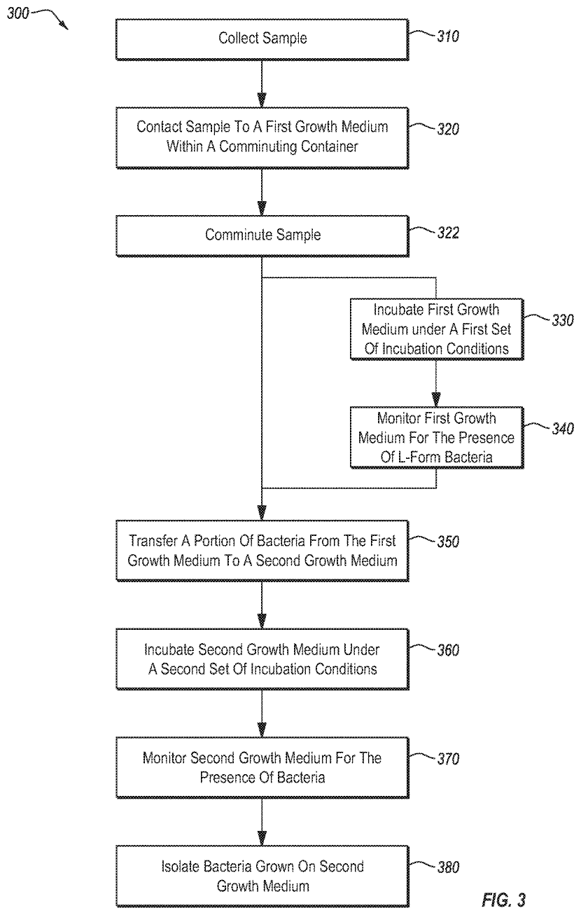

FIG. 3 illustrates another exemplary method 300 of screening for L-form bacteria that includes comminution of the sample. The embodiment shown in FIG. 3 has steps and elements similar to the embodiment shown in FIG. 1, and like numbers represent like elements. As illustrated, the method includes a step 310 of collecting a sample, and a step 320 of contacting the sample to a first growth medium. In some embodiments, the first growth medium is contained within a comminution container. The comminution container is typically formed as an elongate tube with a rounded bottom portion, or with a tapering (e.g., conical frustum) shaped bottom portion.

The comminution container includes a comminuting media configured to contact the sample and disaggregate biofilms, cell clumps, and other aggregates within the sample. The comminuting media is preferably formed from crushed or shattered glass. Other embodiments may include comminuting media formed from beads, shards, particles, fragments, filaments, or other structures configured to contact the sample and disassociate particles within the sample, and may be formed out of metal, plastic, ceramic, or other materials or combinations of materials.

The exemplary method includes a step 322 of comminuting the sample. In some embodiments, the sample and first growth medium are vortexed (e.g., by placing the comminuting container in a vortex apparatus) to displace the comminuting media within the liquid and to enable contact between the comminuting media and the aggregated portions of the sample. In other embodiments, the sample may be comminuted using magnetic stirring (e.g., one or more magnetic stir bars included in the comminuting media), or by shaking, vibrating, or otherwise displacing the comminuting media.

In some embodiments, after comminuting, the method includes a step 330 of incubating the inoculated first growth medium under a first set of incubation conditions and a step 340 of monitoring the inoculated first growth medium for the presence of L-form bacteria. Alternatively, after comminuting, the method can proceed to a step 350 of transferring a portion of the first growth medium to a second growth medium (preferably a solid growth medium) without prior incubation of the sample. Such embodiments can beneficially reduce the culture time required before bacteria can be isolated, analyzed, and/or harvested. For example, the progression of infected red blood cells shown in FIG. 2 can be effectively bypassed or made to progress more rapidly.

In some embodiments, the method then proceeds through a step 360 of incubating the second growth medium under a second set of incubation conditions, a step 370 of monitoring the second growth medium for the presence of bacteria, and optionally a step 380 of isolating bacteria grown on the second growth medium, as described above.

### Inoculant Transfer

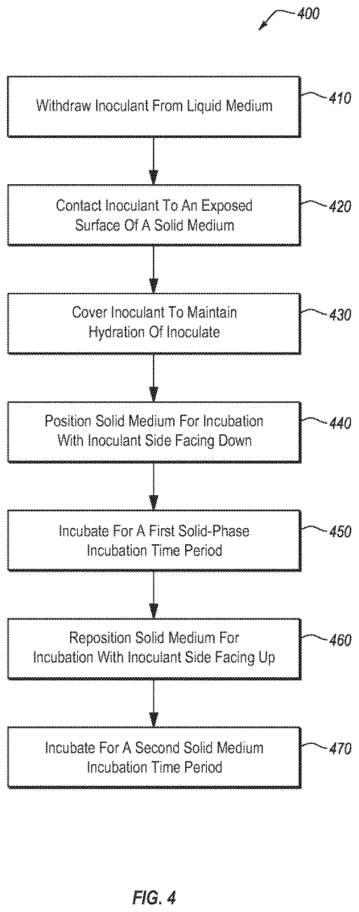

FIG. 4 illustrates an exemplary method 400 for transferring an inoculant from a first, liquid growth medium to a second, solid growth medium and incubating the solid growth medium (e.g., as part of the steps 150 and 160 in the embodiment of FIG. 1 or the steps 350 and 360 in the embodiment of FIG. 3). As shown, the method includes a step 410 of withdrawing an inoculant from the liquid medium, and a step 420 of contacting the inoculant to a surface of a solid medium.

After contacting the inoculant to the solid medium, the method includes a step 430 wherein the inoculant is immediately (e.g., within seconds or within about 1 or 2 minutes) covered by an insert in order to maintain a hydrated state of the inoculant. It has been found that positioning the insert over the inoculant beneficially enables L-form bacteria within the inoculant to interface with the solid substrate to begin colonization of the solid medium. It is theorized that L-form bacteria are often in a hydraulically fragile state at this point in culturing (e.g., due to reduced or absent cell wall structures), and that excessive drying and/or too rapid concentrating of solutes within the inoculant containing the L-form bacteria can inhibit further culturing of the L-form bacteria.

In some embodiments, the insert is a glass panel, glass slide, or other material configured to sit upon the solid media and preferably, to maintain position relative to the solid media (e.g., through adhesive forces between the inner surface of the insert contacting the inoculant and the inoculant). Other embodiments may include inserts made from rigid or film plastics, ceramics, or other materials. Preferably, the insert is positioned to eliminate air pockets within the inoculant between the surface of the solid media and the inner surface of the insert. In some embodiments, an additional amount of inoculant may be contacted to other portions of the surface of the solid media not covered by the insert, if any.

In some embodiments, the method further includes a step 440 of positioning the solid medium for incubation with the inoculant side facing down. For example, where an agarose plate is used to contain the solid media, the plate is positioned “upside down” so that the surface to which the inoculant and insert were applied faces down.

In some embodiments, the method further includes a step 450 of incubating the solid medium for a first solid-phase incubation time period of about 4 hours to 24 hours, or about 6 hours to 18 hours, or about 12 hours. The incubation may be carried out under the temperature conditions described in relation to step 160 of FIG. 1. Preferably, the incubation is also carried out in an atmosphere having a relative humidity that is sufficient to prevent overly rapid drying of the inoculant.

As explained above, it has been discovered that greater culturing efficiency is made possible by maintaining a hydrated state of the inoculants and growth media as the disclosed methods are performed. For example, during the first solid-phase incubation time period, the relative humidity may be maintained within a range of about 40% to about 100%, or about 50% to about 90%, or about 60% to about 80%. In some embodiments, the method further includes a step 460 of repositioning the solid medium with the inoculant side up. It has been discovered that, at this point in the progression of L-form cultures, the L-form bacteria have typically progressed enough and/or the insert has sufficiently interfaced with the solid medium, such that the benefits of repositioning the solid medium to allow evaporation of water that has built up in the inverted position outweigh the detrimental effects, if any, of repositioning.

In some embodiments, the method further includes a step 470 of incubating the solid medium for a second solid-phase incubation period. The second solid-phase incubation time period is preferably performed in an atmosphere having similar relative humidity levels of the first solid-phase incubation time period, and for a time period ranging from about 12 hours to about 84 hours, or about 24 hours to about 72 hours, or about 36 hours to about 60 hours, or about 48 hours. In some embodiments, one or more cultures are further incubated at a temperature in a range that is below body temperature (e.g., about 25° C. to about 35° C., or about 25° C. to about 30° C., or about 27° C.) for a time period of about 4 to about 30 days, or about 7 to 21 about days, or about 14 days. In preferred embodiments, the one or more cultures are further incubated at a temperature below body temperature for a period of about 1 to 7 about days, or about 3-5 days.

In some embodiments, a dual track culturing setup is followed by subjecting a first set of sample portions to a short-track monitoring period and a second set of sample portions to a long-track monitoring period, where the short and long-track monitoring periods have durations according to the above ranges, with the short-track duration being shorter than the long-track duration. Such a dual-track setup has shown good results by enabling faster results from the short-track (e.g., about 1 to about 7 days or about 3 to about 5 days), when possible, without missing the detection of other, slower species and/or strains resulting from the long-track (e.g., about 7 to about 21 days, or about 14 days).

### EXAMPLES

### Example 1

Over 500 separate blood samples were collected (about 120 of these were from individuals who gave more than one sample). More than 30 separate synovial fluid samples and 1 lymphatic fluid sample were also collected. For each sample, about 0.5 ml or less of the sample (about 2 drops) was added to a tube containing 10-15 ml of bovine serum and a tube containing 10-15 ml of BHI broth. The inoculated tubes were incubated at 27° C. Development of L-form culture was monitored by preparing wet mount live slides daily. Samples were monitored for a period of up to 30 days. Samples that showed indications of L-form bacterial growth were typically incubated for at least 48 hours, and typically began to show signs of progressive growth within 48-72 hours. L-form bacteria were not observed to progress to a CWS form while within the broth.

For samples in which L-form bacterial growth was detected, the broth was used to inoculate a variety of agarose plates (mannitol salt, BHI, tryptic soy, tryptic soy w/5% sheep's blood, chocolate blood, Vogel Johnson, Simmons citrate, Columbia, brewer's yeast, nutrient, MacConkey agar, starch agar, and Kligler Iron agar). The inoculant was immediately covered with a sterile cover slide to prevent dehydration of L-form bacteria. Extra inoculant was streaked onto remaining portions of the agarose surface. A set of plates was then incubated at 37° C. in an aerobic incubation unit, and a set of plates was incubated at 37° C. in an anaerobic chamber. Sterile water was supplied in order to maintain a humid environment within the incubation areas. The plates were placed agarose-side down for 12 hours and then were flipped to agarose-side up and incubated for an additional 48 hours. Plates were then removed and sealed in a plastic bag in order to retain moisture and were further incubated at 27° C. for 5 days. At 5 days, plates were inspected for growth. Each colony was transferred (isolated) to a set of nutrient agar and blood agar (trypticase soy agar with 5% sheep blood) plates.

Isolated colonies were characterized using a BioLog GEN III MicroPlate 96 well plate as well as 16S rDNA sequencing. The L-form growth protocols have resulted in the culture and isolation of over 254 unique strains of bacteria originally residing in respective samples as L-form bacteria.

### Example 2

A comparative study was conducted to compare a standard culturing process to the process of Example 1. Each sample was divided into two portions. The first portion was used to directly inoculate two nutrient agars, which were then incubated and monitored for growth. The second portion was used as inoculant in the L-form growth protocol of Example 1. Results of the comparative study are shown in Table 1 (samples which showed no growth in either protocol are omitted).

TABLE 1

| | Bacteria | |
|---|---|---|
| | cultured | Bacteria cultured |
| Sample | via direct | via process of |
| Type | inoculation | Example 1 |
| Blood | No growth | Acintobacter genomospecies |
| | | 15tu |
| | | Bacillus pumilus/safensis |
| | | Bordetella parapertussis |
| | | Simplicispira metamorpha |
| | | Micrococcus luteus A |
| | | Bacillus salentarsenatis/jeotigaii |
| | | Moraxella canis |
| | | Unknown Rod |
| Blood | Bacillus pumilus/ | Bacillus pumilus/safensis |
| | safensis | Bacillus pumilus/safensis |
| | Staph. capitis ss | Bacillus pumilus/safensis |
| | urealyticus | Bacillus thuringiensis/cereus |
| | | Bacilus Vallismortis/subtilis |
| Blood | No growth | Bacillus plakortidis |
| | | Brachybacterium sacelii (26C) |
| | | Unknown Bacteria |
| Blood | No growth | Bacillus pumilus/safensis |
| Blood | No growth | Bacillus pumilus/safensis |
| Blood | No growth | Bacillus lichenformis |
| | | Bacillus lichenformis |
| | | Staphylococcus intermedius |
| Blood | No growth | Bacillus pumilus/safensis |
| Blood | No growth | Bacillus pumilus/safensis |
| | | Micrococcus luteus B |
| | | Corynebacterium terpenotabidum |
| Blood | No growth | Bacillus pumilus/safensis |
| Blood | No growth | Unknown rod |

As shown, growth and culture of L-form bacteria to identifiable classic form was achieved using the process of L-form growth protocol of Example 1, even for many samples which gave no results and no growth under a standard direct inoculation technique. The results show that use of the L-form growth protocol can significantly improve the ability to screen for and then culture and produce L-form capable bacteria.

### Example 3

Of the subjects tested using the culturing process described in Example 1, 10 subjects were found to harbor the Microbacterium maritypicum strain DSM 12512. Of the 10 subjects with this Microbacterium strain, 8 were female, and 7 of the 8 had indicated in a health questionnaire that they suffered from endometriosis or fibroid tumors of the ovaries.

### Example 4

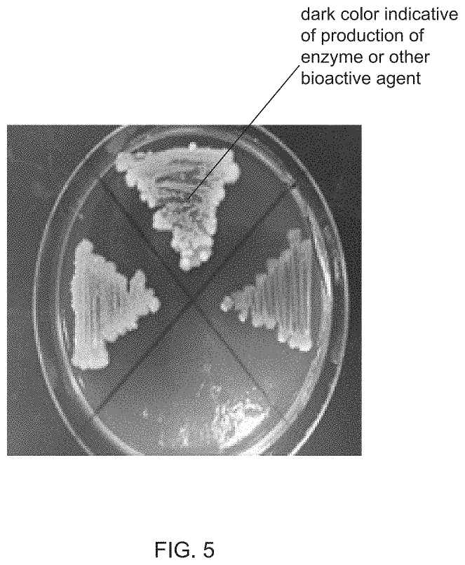

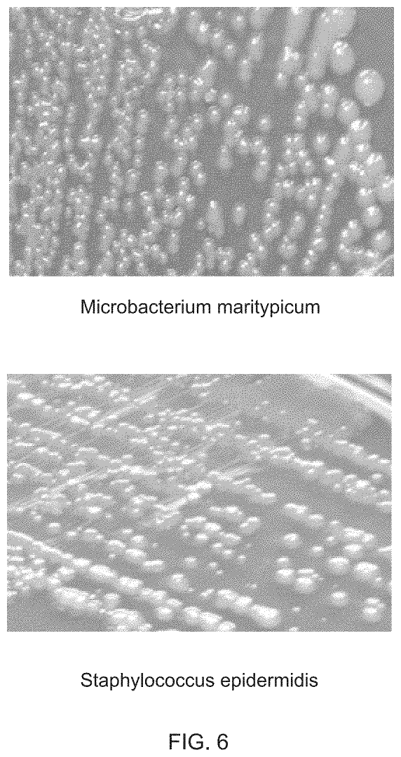

The culture obtained from one of the 7 female subjects self-identifying as having endometriosis was further examined. As shown in FIG. 5, the culture obtained from the subject's sample produced a visible purple hue thought to be indicative of production of one or more biologically active agents such as an enzyme. Genetic analysis showed the culture to be a mixed pair of Microbacterium maritypicum and Staphylococcus epidermidis, shown in FIG. 6. Surprisingly, upon isolation, neither strain produced the purple hue. When reunited as a co-culture, the purple hue returned.

Each of the Microbacterium maritypicum cultures obtained from each of the 7 female subjects who reported suffering from endometriosis or ovarian fibroid tumors were found to produce a purple or orange pigment when co-cultured with any other L-form capable bacteria isolated from the subject (which group consisted of Staphylococcus and Bacillus species).

The culture from the one female subject who was found to harbor L-form Microbacterium maritypicum, but who did not indicate suffering from endometriosis or ovarian fibroid tumors, was also tested in co-culture. Surprisingly, none of the mixed cultures from this subject produced a pigment; however, when the subject's Microbacterium maritypicum was cultured with a Staphylococcus or Bacillus strains from any of the 7 other subjects, the mixed culture did exhibit production of a pigment.

### Example 5

Biological samples (blood samples) were obtained from 16 subjects. Each subject exhibited one or more signs or symptoms of endometriosis and had been previously diagnosed with endometriosis by conventional examination (e.g., physical exam, laparoscopy, etc.). Blood samples were also obtained from 12 healthy subjects, having no prior signs, symptoms or previous diagnosis of endometriosis.

Each sample was divided into two portions. The first portion (internal control) was used to directly inoculate two nutrient agars, which were then incubated and monitored for growth (standard direct inoculation). The second portion was used as inoculant in the L-form growth protocol described in Example 1. No bacterial growth was observed in cultures produced from the healthy volunteer samples. Cultures were subjected to 16S ribosomal sequencing in order to identify the bacterial strains present. Results of the comparative study for subjects having signs or symptoms of endometriosis are shown in Table 2.

TABLE 2

| Subject | | 1609 | 1842 | 1888 | 1905 | 1985 | 3025 | 3104 | 3164 | 1619 | 3016 | 1545 | 1642 | 1644 | 1538 | 1636 | 1661 | Total |
|---|---|---|---|---|---|---|---|---|---|---|---|---|---|---|---|---|---|---|
| Bacteria | Bacillus | X | | | | | | | | X | X | | | | X | | X | 5 |
| | safensis | | | | | | | | | | | | | | | | | |
| | Micrococ | X | X | X | | | X | | X | | | | | | | | | 5 |
| | cus | | | | | | | | | | | | | | | | | |
| | luteus | | | | | | | | | | | | | | | | | |
| | Brachyb | X | | | | | | | | | | | | | | | | 1 |
| | acterium | | | | | | | | | | | | | | | | | |
| | hainane | | | | | | | | | | | | | | | | | |
| | nse | | | | | | | | | | | | | | | | | |
| | Bacillus | | X | | | | | | | | | | | | | | | 1 |
| | simplex | | | | | | | | | | | | | | | | | |
| | Paenibac | | X | | | | | | | | | | | | | | | 1 |
| | illus | | | | | | | | | | | | | | | | | |
| | lautus | | | | | | | | | | | | | | | | | |
| | Bacillus | | | X | | | | | | | X | | | | | X | | 3 |
| | lichenifor | | | | | | | | | | | | | | | | | |
| | mis | | | | | | | | | | | | | | | | | |
| | Micrococ | | | X | | X | | | | | | | | | | | | 2 |
| | cus | | | | | | | | | | | | | | | | | |
| | aloeverae | | | | | | | | | | | | | | | | | |
| | Staphylo | | | X | | | | | | | | | | | | | | 1 |
| | coccus | | | | | | | | | | | | | | | | | |
| | petrasii | | | | | | | | | | | | | | | | | |
| | Bacillus | | | | X | | | | | | | | | | | | | 1 |
| | stratosp | | | | | | | | | | | | | | | | | |
| | hericus | | | | | | | | | | | | | | | | | |
| | Bacillus | | | | X | | | | | | | | | | | | | 1 |
| | drentensis | | | | | | | | | | | | | | | | | |
| | Planococ | | | | X | | | | | | | | | | | | | 1 |
| | cus | | | | | | | | | | | | | | | | | |
| | antarcticus | | | | | | | | | | | | | | | | | |
| | Pseudom | | | | X | | | | | | | | | | | | | 1 |
| | onas | | | | | | | | | | | | | | | | | |
| | parafulva | | | | | | | | | | | | | | | | | |
| | Staphylo | X | | | | X | X | | | | | X | | | X | | | 5 |
| | coccus | | | | | | | | | | | | | | | | | |
| | epidermi | | | | | | | | | | | | | | | | | |
| | dis | | | | | | | | | | | | | | | | | |
| | Bacillus | | | | | | X | X | X | X | | | | | X | X | | 6 |
| | subtilis | | | | | | | | | | | | | | | | | |
| | Bacillus | | | | | | X | | | | | | | | | | | 1 |
| | velezensis | | | | | | | | | | | | | | | | | |
| | Kocuria | | | | | | X | | | | | | | | | | | 1 |
| | himachal | | | | | | | | | | | | | | | | | |
| | ensis | | | | | | | | | | | | | | | | | |
| | Staphylo | | | | | | X | | | | | | | | | | | 1 |
| | coccus | | | | | | | | | | | | | | | | | |
| | equorum | | | | | | | | | | | | | | | | | |
| | Staphylo | | | | | | X | | | | | | | | | | | 1 |
| | coccus | | | | | | | | | | | | | | | | | |
| | pasteuri | | | | | | | | | | | | | | | | | |
| | Streptom | | | | | | X | | | | | | | | | | | 1 |
| | yces | | | | | | | | | | | | | | | | | |
| | speibonae | | | | | | | | | | | | | | | | | |
| | Dietzia | | | | | | | X | | | | | | | | | | 1 |
| | maris | | | | | | | | | | | | | | | | | |
| | Microbac | | | | | | | | | X | | | | | | | | 1 |
| | terium | | | | | | | | | | | | | | | | | |
| | barkeri | | | | | | | | | | | | | | | | | |
| | Planococ | | | | | | | | | X | | | | | | | | 1 |
| | cus | | | | | | | | | | | | | | | | | |
| | kocurii | | | | | | | | | | | | | | | | | |
| | Staphylo | | | | | | | | | X | | | | | | | | 1 |
| | coccus | | | | | | | | | | | | | | | | | |
| | warnei | | | | | | | | | | | | | | | | | |
| | Microbac | | | | | | | | | X | X | X | X | X | X | X | X | 8 |
| | terium | | | | | | | | | | | | | | | | | |
| | maritypic | | | | | | | | | | | | | | | | | |
| | um | | | | | | | | | | | | | | | | | |
| | Bacillus | | | | | | | | | | X | | X | | | | | 2 |
| | cereus | | | | | | | | | | | | | | | | | |
| | Bacillus | | | | | | | | | | X | | | | | | | 1 |
| | subterra | | | | | | | | | | | | | | | | | |
| | neus | | | | | | | | | | | | | | | | | |
| | Microbac | | | | | | | | | | X | | | | | | | 1 |
| | terium | | | | | | | | | | | | | | | | | |
| | flavum | | | | | | | | | | | | | | | | | |
| | Amnibac | | | | | | | | | | | | | | | X | | 1 |
| | terium | | | | | | | | | | | | | | | | | |
| | kyonggie | | | | | | | | | | | | | | | | | |
| | nse | | | | | | | | | | | | | | | | | |
| | Bacillus | | | | | | | | | | | | | | | | X | 1 |
| | Persicus | | | | | | | | | | | | | | | | | |
| | Bacillus | | | | | | | | | | | | | | | | X | 1 |
| | thuringie | | | | | | | | | | | | | | | | | |
| | nsis | | | | | | | | | | | | | | | | | |

It was observed that 8 of the 16 subjects tested (50%) were infected with L-form Microbacterium maritypicum and presented with signs and symptoms of endometriosis. Additionally, it was observed that 7 of the 8 subjects (˜87%) that were infected with L-form M. maritypicum were co-infected with at least one L-form Bacillus and/or L-form Staphylococcus bacterial species. No bacterial growth was observed in the internal control samples from each of the endometriosis subjects.

### Example 6

Bacterial strains identified in Example 5 were tested for antibiotic sensitivity by a diffusion disk test. Table 3 describes the most effective intravenous (IV) and oral antibiotic agents tested against each strain. Also described in Table 3 are antibiotic agents that bacterial strains cultured from each subject were observed to display resistance. In the majority of samples, resistance to Metronidazole was observed for M. maritypicum. Resistance to Oxacillin was also observed in the M. maritypicum samples.

TABLE 3

| | Most Effective | Most Effective | |
|---|---|---|---|
| Subject | IV Antibiotic | Oral Antibiotic | Resistance? |
| 1609 | Meropenem | Doxycycline | Metronidazole, |
| | | | Oxaillin, Cefuroxime |
| 1842 | Meropenem | Doxycycline | Metronidazole, |
| | | | Oxacillin |
| 1888 | | Amoxiclav | |
| 1905 | Imipenem | Amoxiclav, | |
| | | Penicillin | |
| 1985 | | Rifapin | Metronidazole |
| 3025 | Imipenem, Meropenem | Amoxiclav | Oxacillin, Clinamycin |
| 3104 | Imipenem, Meropenem | Doxycycline | |
| 3164 | imipenem, Meropenem | Doxycycline | Metronidazole |
| 1619 | Imipenem, Meropenem | Doxycycline | Metronidazole, |
| | | | Oxacillin |
| 3016 | | Amoxiclav | Metronidazole, |
| | | | Oxacilin, Cefuroxime, |
| | | | Sulfa-Trimeth, |
| | | | Penicillin, Clindamycin |
| 1545 | | Doxycycline | Metronidazile, |
| | | | Oxacillin, Sulfa- |
| | | | Trimeth, Clindamycin |
| 1642 | | Doxycycline | Metronidazole, |
| | | | Oxacillin, Cefuroxime, |
| | | | Sulfa-Trimeth, |
| | | | Amoxiclav |
| 1644 | | Cefuroxime | Metronidazole, |
| | | | Oxacillin, Clindamycin |
| 1538 | Imipenem, Meropenem | Amoxiclav | Metronidazole |
| | | | Clindamycin |
| 1636 | Imipenem, Meropenem | Doxycycline | Metronidazole, |
| | | | Clindamycin, Oxacillin |
| 1661 | Imipenem | Doxycycline | Metronidazole, |
| | | | Clindamycin |

### Example 7

Based upon the results of the diffusion disk sensitivity testing described in Example 6, subjects identified as being infected with L-form M. maritypicum are administered the antibiotic agent that is effective against M. maritypicum are not resistant). Preferably, none of the other L-form bacterial strains identified in a given subject (e.g., Bacillus, Staphylococcus, etc.) are resistant to the selected antibiotic. As a result of being administered the antibiotic agent, subjects are expected to have a reduction (or clearance) of L-form bacteria and improvement in one or more signs or symptoms of endometriosis. In some embodiments, subjects that are administered an antibiotic agent have increased fertility relative to their fertility prior to being administered the antibiotic agent.

Although the foregoing has been described in some detail by way of illustrations and examples for purposes of clarity and understanding, it will be understood by those of skill in the art that numerous and various modifications can be made without departing from the spirit of the present disclosure. Therefore, it should be clearly understood that the forms disclosed herein are illustrative only and are not intended to limit the scope of the present disclosure, but rather to also cover all modification and alternatives coming with the true scope and spirit of the invention.

What is claimed is:

1. A method for detecting an L-form bacterial infection in a subject in order to diagnose the subject as having endometriosis and/or an ovarian fibroid tumor, the method comprising:
subjecting a sample obtained from the subject to conditions that promote the culturing of L-form bacteria present within the sample;
detecting the presence or absence of bacteria of the genus Microbacterium as a result of the culturing; and
based on detecting the presence or absence of Microbacterium bacteria, diagnosing the subject as likely having or not having endometriosis and/or an ovarian fibroid tumor.

2. The method of claim 1, further comprising:
based on detecting the presence of Microbacterium bacteria, continuing one or more additional tests for diagnosing endometriosis and/or an ovarian fibroid tumor; or
based on failing to detect the presence of Microbacterium bacteria, foregoing one or more additional tests for diagnosing endometriosis and/or an ovarian fibroid tumor, or continuing further testing that is not specific to diagnosing endometriosis and/or an ovarian fibroid tumor.

3. The method of claim 1 or claim 2, wherein the culturing comprises:
contacting the sample to a first growth medium;
incubating the first growth medium under a first set of incubation conditions;
transferring at least a portion of the first growth medium, as an inoculant, to a second growth medium under conditions that maintain a hydrated state of the inoculant; and
incubating the second growth medium under a second set of incubation conditions.

4. The method of any one of claims 1 through 3, wherein the Microbacterium bacteria is Microbacterium maritypicum.

5. The method of any one of claims 1 through 4, further comprising detecting the presence of at least one other L-form bacteria that is an antagonist or symbiont with the Microbacterium bacteria, and based on detecting the presence of the Microbacterium bacteria and the at least one other L-form bacteria, diagnosing the patient as having or likely having endometriosis and/or an ovarian fibroid tumor.

6. The method of claim 6, wherein the at least one other L-form bacteria is a Staphylococcus or Bacillus species.

7. The method of any one of claims 1 through 6, wherein the method has an accuracy for positive results that is higher than about 50%, higher than about 60%, higher than about 70%, higher than about 80%, or higher than about 85%.

8. The method of any one of claims 1 through 7, wherein the first growth medium is a liquid, and wherein the second growth medium is a solid.

9. The method of claim 8, wherein the inoculant is added to a surface of the second growth medium, the method further comprising placing an insert over the inoculant to seal at least a portion of the inoculant between the surface of the second growth medium and the insert.

10. The method of claim 9, wherein the second set of incubation conditions includes a first solid-phase incubation time period and a second solid-phase incubation time period, the second growth medium being positioned inoculant side down for the first solid-phase incubation time period and inoculant side up for the second solid-phase incubation time period.

11. The method of any one of claims 1 through 10, wherein the first and second growth media are independently selected from the group consisting of: mannitol salt, Kligler iron, Vogel Johnson, Columbia blood, brain heart infusion (BHI), nutrient, bovine serum, and human serum.

12. The method of any one of claims 1 through 11, wherein at least one of the first and second growth media is a complex media.

13. The method of any one of claims 1 through 12, further comprising comminuting the sample prior to transferring the sample to the second growth medium in order to increase culture growth and/or decrease culture time.

14. The method of claim 13, wherein comminution is performed in a comminuting container containing a comminuting media, the comminuting media being configured to contact portions of the sample to increase exposure of L-form bacteria within the sample to surrounding growth media.

15. A method for treating endometriosis in a subject, the method comprising obtaining or having obtained a biological sample from the subject; detecting or having detected in the biological sample one or more L-form bacteria, each L-form bacteria having a genus selected from Microbacterium, Bacillus, Micrococcus, and Staphylococcus; and administering to the subject a therapeutically effective amount of an antibiotic agent based upon the presence of the one or more bacteria in the biological sample.

16. The method of claim 15, wherein the subject has or is suspected of having endometriosis, optionally wherein the subject is characterized by one or more signs, symptoms, or risk factors associated with endometriosis.

17. The method of claim 15 or 16, wherein the subject is a human.

18. The method of any one of claims 15 to 17, wherein the biological sample is blood.

19. The method of any one of claims 15 to 18, wherein the detecting comprises the method of any one of claims 1 to 14.

20. The method of any one of claims 15 to 19, wherein at least one of the L-form bacteria is of a species set forth in Table 2.

21. The method of any one of claims 15 to 20, wherein the Micrococcus is Micrococcus luteus.

22. The method of any one of claims 15 to 20, wherein the Bacillus is Bacillus safensis, Bacillus simplex, Bacillus licheniformis, Bacillus stratosphericus, Bacillus dretensis, Bacillus subtillis, Bacillus velezensis, Bacillus cereus, Bacillus subterraneus, Bacillus persicus, or Bacillus thuringiensis.

23. The method of any one of claims 15 to 20, wherein the Microbacterium is Microbacterium maritypicum.

24. The method of any one of claims 15 to 23, wherein the detecting comprises detecting or having detected one or more additional L-form bacteria in the biological sample.

25. The method of claim 24, wherein the one or more additional L-form bacteria are of a genus selected from Staphylococcus, Brachybacterium, Paenibacillus, Planococcus, Pseudomonas, Kocuria, Streptomyces, Dietzia, and Amnibacterium.

26. The method of claim 25, wherein the Staphylococcus is Staphylococcus epidermis, Staphylococcus petrasii, Staphylococcus equorum, Staphylococcus pasteuri, Staphylococcus speibonae, and Staphylococcus warneri.

27. The method of any one of claims 15 to 26, wherein the antibiotic agent is a broad-spectrum antibiotic, optionally wherein the broad-spectrum antibiotic is a beta-lactam antibiotic.

28. The method of claim 27, wherein the broad-spectrum antibiotic is a carbapenem, optionally wherein the carbapenem is Imipenem or Meropenem.

29. The method of claim 27, wherein the broad-spectrum antibiotic is a tetracycline, optionally wherein the tetracycline is Doxycycline.

30. The method of claim 27, wherein the broad-spectrum antibiotic is Amoxiclav.

31. The method of claim any one of claims 15 to 30, wherein the antibiotic agent is administered intravenously.

32. The method of any one of claims 15 to 30, wherein the antibiotic agent is administered orally.

33. The method of any one of claims 15 to 32, wherein the antibiotic agent is administered to the subject during menstruation of the subject.
